(19) United States
(12) Patent Application Publication
(10) Pub. No.: US 2026/0069452 A1
(43) Pub. Date: Mar. 12, 2026

(54) Fractionated Drug Delivery of Biological or Chemical Agents

(71) Applicant: Bausch + Lomb Ireland Limited, Dublin (IE)

(72) Inventors: Mark S. Blumenkranz, Portola Valley, CA (US); Yehuda Ivri, Newport Coast, CA (US); Reynaldo Quintana, Menlo Park, CA (US); Daniel V. Palanker, Sunnyvale, CA (US)

(21) Appl. No.: 19/386,603

(22) Filed: Nov. 12, 2025

Related U.S. Application Data
(63) Continuation of application No. 17/096,348, filed on Nov. 12, 2020, now Pat. No. 12,496,218.
(60) Provisional application No. 62/934,225, filed on Nov. 12, 2019.
(60) Provisional application No. 62/951,903, filed on Dec. 20, 2019.
(60) Provisional application No. 63/011,800, filed on Apr. 17, 2020.
(60) Provisional application No. 63/011,808, filed on Apr. 17, 2020.
(60) Provisional application No. 63/049,110, filed on Jul. 7, 2020.
(60) Provisional application No. 63/049,582, filed on Jul. 8, 2020.

(51) Int. Cl. A61F 9/00; G16H 20/13; G16H 40/67

(52) U.S. Cl. CPC A61F 9/0008; G16H 20/13; G16H 40/67

(57) ABSTRACT

The efficacy of a dosing regimen can be altered, and preferably improved, by fractionating the doses. Here fractionating the doses can be regarded as taking a single conventional dose and splitting that dose into two or more dose-fractions delivered at relatively short time intervals, where the dose-fractions add up to the same total dose as the conventional dose. In cases where dose fractionation increases the therapeutic effect, this can result in a greater therapeutic effect from a smaller total dose when the doses are fractionated. This effect has been demonstrated in an ophthalmic application, but is expected to be more generally applicable.

### CROSS REFERENCE TO RELATED APPLICATIONS

This application is a continuation of U.S. patent application Ser. No. 17/096,348, entitled “Fractionated Drug Delivery of Biological or Chemical Agents,” filed Nov. 12, 2020, which is hereby incorporated by reference in its entirety.

This application claims the benefit of U.S. provisional patent application 62/934,225, filed on Nov. 12, 2019, and hereby incorporated by reference in its entirety.

This application claims the benefit of U.S. provisional patent application 62/951,903 filed on Dec. 20, 2019, and hereby incorporated by reference in its entirety.

This application claims the benefit of U.S. provisional patent application 63/049,582, filed on Jul. 8, 2020, and hereby incorporated by reference in its entirety.

This application claims the benefit of U.S. provisional patent application 63/011,800, filed on Apr. 17, 2020, and hereby incorporated by reference in its entirety.

This application claims the benefit of U.S. provisional patent application 63/049,110, filed on Jul. 7, 2020, and hereby incorporated by reference in its entirety.

This application claims the benefit of U.S. provisional patent application 63/011,808, filed on Apr. 17, 2020, and hereby incorporated by reference in its entirety.

### FIELD OF THE INVENTION

This invention relates to dosing of biological or chemical agents for therapeutic effects.

### BACKGROUND

Dose optimization for therapy using chemical or biological agents has been extensively investigated. The usual parameters subject to optimization are the size of each dose and the timing between doses. This dose timing is typically at regular intervals, accommodating for the sleep-wake cycle as needed.

### SUMMARY

We have found, unexpectedly, that the efficacy of a dosing regimen can be altered, and preferably improved, by fractionating the doses. Here fractionating the doses can be regarded as taking a single conventional dose and splitting that dose into two or more dose-fractions delivered at relatively short time intervals, where the dose-fractions add up to the same total dose as the conventional dose. Our experimental data show this effect in an ophthalmic application, but it is expected that this effect can occur more generally.

More specifically, a fractionated dosing regimen entails the splitting of a dose into two or more dose-fractions separated by one or more dose-fraction time intervals in a range from 30 seconds to 30 minutes. These dose-fraction time intervals need not be the same for each consecutive pair of dose-fractions.

It is convenient to refer to such a split dose as a “fractionated-dose” having a total administered dose that is the sum of the doses provided by its dose-fractions.

In some embodiments, two or more such fractionated-doses are separated by one or more fractionated-dose time intervals of an hour or more. These fractionated-dose time intervals need not be the same for each consecutive pair of fractionated-doses.

Thus the fractionated-doses are the doses that can have a ‘typical’ delivery schedule, such as every 4 hours, 5 times a day or the like, and the core idea is that each one of these fractionated-doses is split into the dose-fractions to provide fractionated dosing.

The biological effect (or effects) of a therapeutic agent can be different with fractionated dosing vs. unfractionated dosing. Our results show that in some cases a desirable therapeutic effect is enhanced by fractionated dosing. In cases where the agent has multiple effects, some effects can be increased by fractionation, other effects may be decreased by fractionation, and further effects may be unchanged by fractionation.

### BRIEF DESCRIPTION OF THE DRAWINGS

FIG. 1 schematically shows an example of conventional dosing.

FIG. 2 schematically shows an example of fractionated dosing.

FIG. 3 shows experimental visual acuity results comparing conventional dosing to fractionated dosing.

FIG. 4 shows further experimental visual acuity results comparing conventional dosing to fractionated dosing.

FIG. 5 shows experimental intraocular pressure results comparing conventional dosing to fractionated dosing.

FIG. 6 shows a first example of a preferred handheld dispenser for topical ocular medications.

FIG. 7 shows a second example of a preferred handheld dispenser for topical ocular medications.

### DETAILED DESCRIPTION

Section A describes general principles relating to embodiments of the invention. Section B describes experimental evidence for the effect of dose fractionation. Section C describes several variations relating to preferred embodiments.

### A) General Principles

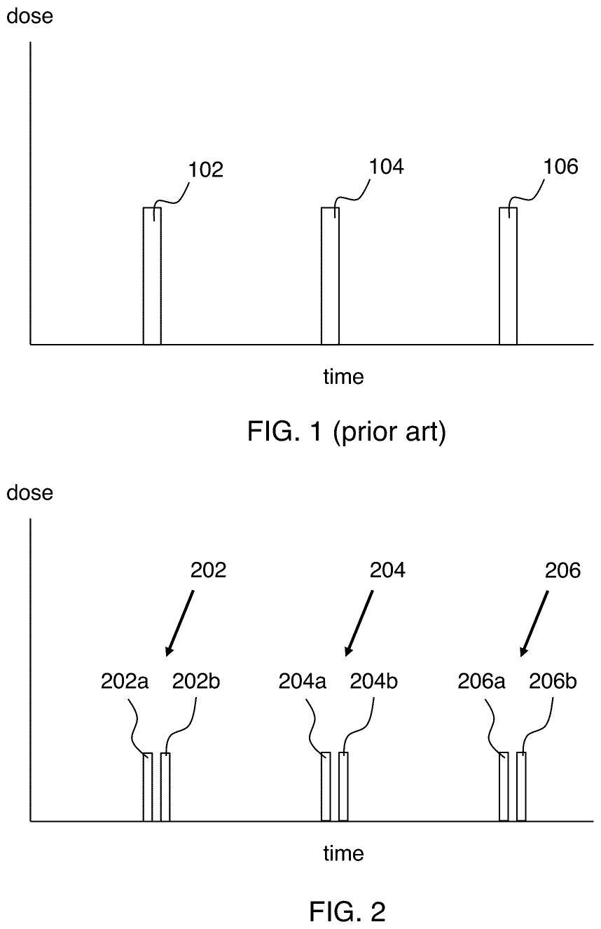

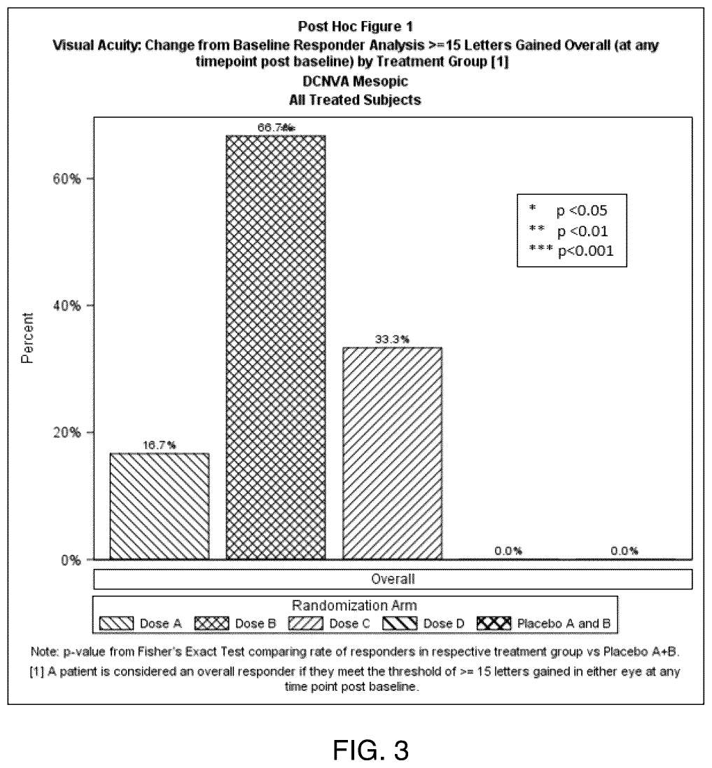

FIG. 1 schematically shows a conventional dosing regimen. Here dosing vs. time is shown for three conventional doses 102, 104, and 106. FIG. 2 schematically shows an example of fractionated dosing. Here a first fractionated-dose 202 is split into two dose-fractions 202a and 202b. Similarly, a second fractionated-dose 204 is split into two dose-fractions 204a and 204b, and a third fractionated-dose 206 is split into two dose-fractions 206a and 206b.

As described in more detail below, we have unexpectedly found, in studies of topical ocular medications in humans, that such dose fractionation can have different (and sometimes better) therapeutic effects compared to conventional single-dosing having the total dose of the dose-fractions delivered in a single dose.

Accordingly, a first embodiment of the invention is a method of delivering a dose of a therapeutic biological or chemical agent to an ocular surface of an eye of a patient, where the method includes delivering the therapeutic biological or chemical agent to the ocular surface of the eye of the patient by fractionating the dose into two or more dose-fractions, and the dose-fractions are separated by one or more dose-fraction time intervals in a range from 10 seconds to 30 minutes.

This first embodiment can further include increasing a therapeutic effect of the therapeutic biological or chemical agent in humans by the dose fractionation.

A second embodiment of the invention is a method of delivering a dose of a therapeutic biological or chemical agent to a patient, where the method includes i) delivering the therapeutic biological or chemical agent to the patient by fractionating the dose into two or more dose-fractions, and the dose-fractions are separated by one or more dose-fraction time intervals in a range from 10 seconds to 30 minutes; and ii) altering one or more biological effects of the therapeutic biological or chemical agent in the patient by the fractionating the dose into two or more dose-fractions.

In this second embodiment, a therapeutic effect of the therapeutic biological or chemical agent in humans can be increased by the dose fractionation.

In both embodiments, the dose-fraction time intervals are preferably in a range between 1 minute and 10 minutes. The therapeutic biological or chemical agent can be a liquid ophthalmic agent, and the dose-fractions can be micro-doses having a volume in a range between 1 □ l and 20 l □ l. Such micro-doses have the advantage of more precise drug delivery to the eye, since their volume can be accommodated by the eye. In contrast, conventional eyedroppers often lead to imprecise dosing because the volume of the dose is too large to be accommodated in the eye and the excess liquid spills out, leading to poor knowledge of the actual delivered dose.

In both embodiments, a fractionated dosing regimen can be used where the fractionated-doses described above are delivered at two or more times separated by one or more fractionated-dose time intervals of an hour or more.

### B) Experimental Evidence

FIG. 3 shows visual acuity results for various dosing regimens of pilocarpine. Here dose regimen A is single dosing of 1% pilocarpine, dose regimen B is double dosing of 1% pilocarpine, dose regimen C is single dosing of 2% pilocarpine, and dose regimen D is double dosing of 2% pilocarpine. Placebo A and B are single and double doses of saline, respectively. According to the design of this study, dose regimen B is a fractionated dosing regimen relative to the conventional single dosing regimen of dose regimen C, with a dose-fraction time interval of 5 minutes. In other words, the double dosing (fractionation) of dose regimen B provides the same total dose (drug mass) applied to the eye as the single (non-fractionated) dosing of regimen C.

A significant portion (66.7%) of subjects treated with a fractionated microdose of 1% pilocarpine (dose regimen B) gained more than 15 letters of distance corrected near vision compared with baseline distance corrected near visual acuity under both mesopic and photopic illumination conditions. Despite being the same total amount of active drug, the fractionated micro-dose of 1% pilocarpine (dose regimen B) performed significantly better than 1 dose of 2% pilocarpine (dose regimen C) despite the fact that the total amount of drug applied to the surface of the eye within that five minute time interval was the same in all testing conditions (mesopic and photopic lighting conditions). Additionally, double dosing of 2% pilocarpine (dose regimen D) was less effective; 0% of eyes achieved 3 lines of distance corrected near vision improvement versus 33.3% of eyes receiving a single non fractionated dose of 2% pilocarpine in the absence of any evident toxic effects. However compared with a single conventional dose of 1% pilocarpine (group A), a double (fractionated) dose of 1% pilocarpine (group B) resulted in statistically significant improvement in distance corrected near visual acuity.

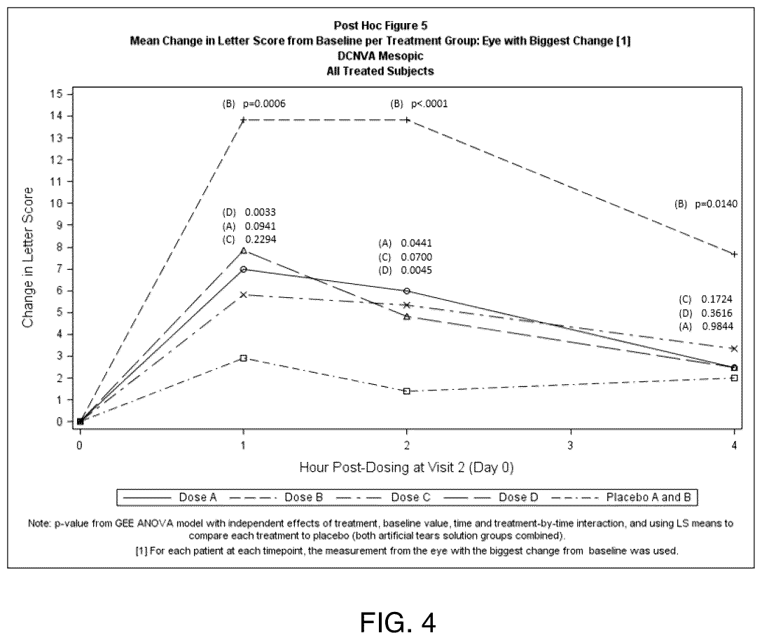

Therefore the results of fractionated dosing are non-linear and non-obvious and dependent on the specific concentration and mechanism of action of the drug used and may be either beneficial or adverse or undetectable as the case may be. Change from baseline DCNVA (distance corrected near visual acuity) was also analyzed per treatment group and time point for both eyes in photopic conditions, as presented in FIG. 4. Again, fractionated micro-dosing with 1% pilocarpine (dose regimen B) resulted in the biggest gain from baseline at all time points. Post hoc analyses showed that these gains in letter score were statistically significantly different from baseline at 1 and 2 hours post-dosing (hour 1 and 2: p<0.001). It is clear that fractionated micro-dosing has increased efficacy over similar amounts of drug (and sometimes higher and lower amounts of drug) at the proper dosing level.

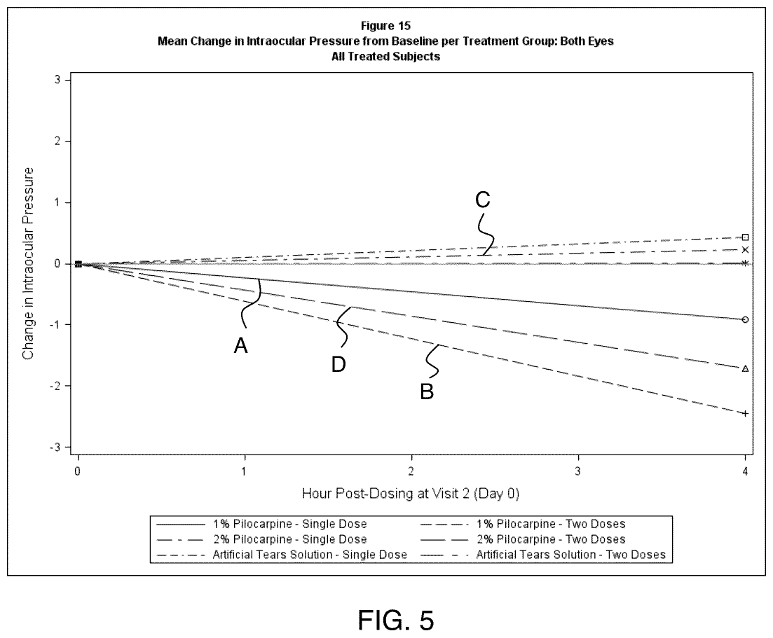

FIG. 5 shows IOP (intraocular pressure) results for the dosing regimens identified above. Again, dosing regimen B (fractionated dosing) performed much better than dosing regimen C (corresponding comparable total dose, not fractionated). However, the IOP results differ from the above visual acuity results in other respects. A particularly noteworthy feature is the null IOP result for dosing regimen C, which is not presently understood. The bottom line appears to be that different therapeutic effects can respond differently to dose-fractionation, even for the same therapeutic agent.

The above results confirm the utility of aspects of the present invention of “fractionated dosing”, where more than one lower concentration dose is administered according to a spaced out over an interval of time regimen, resulting in greater effect than a higher concentration dose (or series of doses). This phenomenon is at least in part due to the fact that there is often a concentration dependence to certain effects and side effects, where at lower concentrations a desired effect is seen but with fewer undesired effects, but at higher concentrations, the same effect is seen but with more or new undesired effects. Such undesired effects may not necessarily be adverse effects, just effects that are not desired within a particular context. In the present case, lower concentration pilocarpine given at two doses separated in time had a greater effect on near vision than high concentration pilocarpine at a single or two doses spaced over the same interval. This result suggests that higher concentration pilocarpine exerts certain biological effects outside of pupillary constriction that may interfere with visual-benefits of pupillary constriction, while the lower concentration pilocarpine exerts effects more limited to pupillary constriction and, in turn, near visual acuity. Other types of duty cycles of multiple dosing regimens are possible in this case, including more than two doses separated in time and time intervals of varying duration between the doses.

The discovery that two fractionated doses of an active miotic agent were more effective in achieving a miotic benefit in humans than one dose of a similar concentration is unexpected since our experiments in rabbits do not support that expectation. Additionally our finding that two doses of a higher concentration of cholinergic agent may provide similar or improved miosis yet worsened near visual acuity benefit is also unexpected as demonstrated in our human experiments and indicate an additional mechanism contributing to vision gain or benefit beyond simply pupil size including possible effects on accommodation, position of the lens iris diaphragm, intraocular pressure, neural transduction or other possibilities relating to differential activation of different receptor classes or neuro-physiologic effects for different doses of drug.

Embodiments of fractionated dosing described herein provide a continuous low dose of a drug in which there are discrete separations between low dosage pulses rather than a continuous stream. Literature data (see Kompella et al., “ISOPT Clinical Hot Topic Panel Discussion on Ocular Drug Delivery,” J. Ocul. Pharmacol. Ther. (2019) 35(8): 457-465) has shown that the anterior chamber concentration of certain drugs reaches a maximum at around 5 minutes. Meanwhile, the ocular surface can only hold about 7-8 microliters at a time but is continuously turned over as tears are produced and drained through the lacrimal system. Therefore, administration of a second dose after a period of about 5 minutes would not only replenish the drug at the ocular surface, but also allow concentration in the anterior chamber again as the intracameral concentration of the drug has begun to decline. In certain embodiments, the second dose is given after the drug is completely cleared from the target tissue (or anatomic space in the body); in other embodiments, it is given when the drug is only partially cleared from the target tissue. In still others, it is given before it has reached its maximal concentration.

For embodiments of the present invention that administer only a small quantity of drug, the amount of bioavailable drug following 2 or more fractionated micro-doses may counter-intuitively provide for greater bioavailability and pharmacodynamics even though the total cumulative dose may only be half of that with one conventional drop. Finally there may also be receptor occupancy effects whereby with the passage of a certain period of time, there may be more than one cycle of activation coupled with fewer off target effects from the larger cumulative drugs not just to the corneal surface but to surrounding tissues in the ocular adnexa, such as the outer lid surfaces, other glands, and tissues that may be impacted by nasolacrimal drainage and reflux. It has been taught that for the case of muscarinic receptors initial binding of cholinergic agonists may allosterically affect the conformation of the M3 and related receptors such that there is a change in the access to or affinity for other binding sites on the receptor for subsequent interaction with cholinergic agents. [Mol Pharmacol. 2017 Nov; 92(5): 601-612. “Teaching an Old Drug New Tricks: Agonism, Antagonism, and Biased Signaling of Pilocarpine through M3 Muscarinic Acetylcholine Receptor”, Alexey N. Pronin, Qiang Wang, and Vladlen Z. Slepak, hereby incorporated by reference in its entirety].

This enhanced pharmacological effect with fractionated drop administration is not limited to the treatment of presbyopia but may also be used with oculo-hypotensive agents, steroids, non-steroidal agents, dry eye treatments, antibiotics, mydriatics, and the like with different targeted receptor types and corresponding classes of chemical and biologic agents beyond muscarinic receptors and cholinergic agents. In addition, the short-interval, fractionated treatments disclosed herein may overcome the need for viscosity enhancing agents that increase retention time on the ocular surface (e.g. hydroxypropyl methylcellulose (HPMC)), and also overcome issues inherent to certain sustained drug release technologies where this continuous delivery of a drug and no time interval allowed for the local tissue environment to return to equilibrium (where the drug has been partially or completely cleared from the target tissue or anatomic space.)

### C) Variations

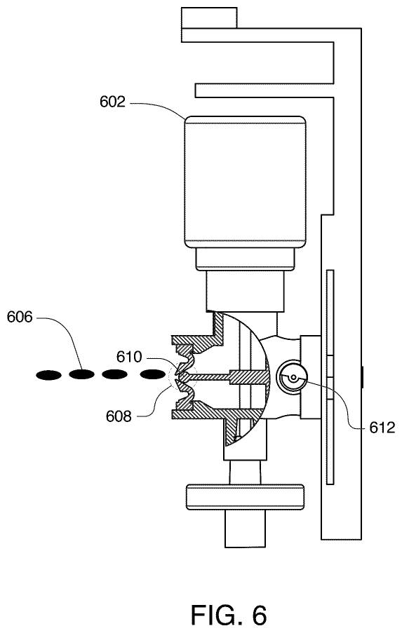

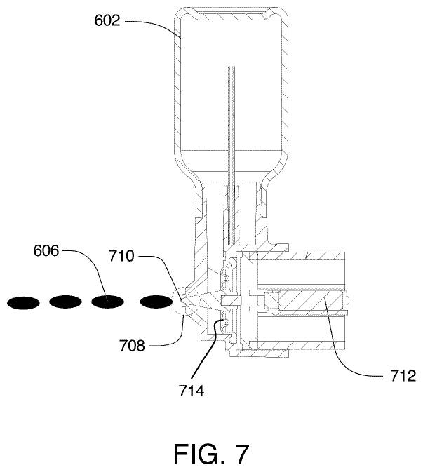

In preferred embodiments, the therapeutic agent being delivered is a liquid ophthalmic agent that is dispensed using a handheld dispenser. FIG. 6 schematically shows a first example of a suitable handheld dispenser. In the example of FIGS. 6, 602 is the drug package, 606 is the emitted fluid, 610 is the valve that keeps aperture 608 normally closed, and 612 is the vibrational motor responsible for generating pressure pules. The example of FIG. 7 is similar, except that valve 710 and aperture 708 have a different configuration, as does the motor 712. The emitted fluid in 606 is depicted as a discontinuous stream of fluid droplets but may also be a continuous stream of fluid droplets, for example when the tail of each drop collides with the head of the following drop. The key difference between the embodiment in FIG. 6 and that of FIG. 7 is that in the FIG. 6. embodiment the vibrating motor is vibrating the entire assembly whereas in the embodiment of FIG. 7 the vibrating motor is only vibrating the membrane 714.

The system is configured to dispense fluid by generating pressure pulses by way of volume displacement or by generation of acoustic pulses from the vibration motor. The pulses may also be generated via a piezoelectric or electromagnetic transducer. In one embodiment the pulses are generated by a vibrational DC motor. The oscillation of the vibrational motor may be transmitted to the fluid through a membrane 714 (FIG. 7) or by oscillating the entire drug fluid package (FIG. 6).

The drug is dispensed from an aperture as a stream of micro-droplets when one or more pulses is exerted onto the fluid in the drug package.

The drug or fluid package can be a blow-fill-seal package or an ampoule containing an ophthalmic formulation.

The drug or fluid package may also include a valve that is configured to normally seal the opening of the aperture to prevent microbial contamination of the fluid within the drug package. The valve opens strictly when fluid is dispensed.

Dose volumes are generally between 7-15 ul. Such volumes generally remain on the eye, however in some embodiments the device is configured to dispense 30-40 ul, which is comparable to a squeeze bottle eye dropper.

The device can further include an electronic circuit that is configured to generate and transmit an electric pulse or wave form to the motor. The circuit may include a timer circuit which determines the length of time that is required to deliver the dose volume.

Accordingly, preferred embodiments of the methods described above include use of a handheld dispenser to provide the liquid ophthalmic agent to the eye(s) of the patient. The handheld dispenser can include: a container configured to hold the liquid ophthalmic agent and having an aperture, where the dispenser is configured to emit the dose-fractions from the container through the aperture.

Various additional features can be provided by the handheld dispenser. The handheld dispenser can be configured to provide a dose-fraction signal when a next dose-fraction is due to be delivered. Similarly, the handheld dispenser can be configured to provide a fractionated-dose signal when a next fractionated-dose is due to be delivered.

The handheld dispenser can be in wireless communication with one or more remote devices to provide functions including but not limited to: remote dose monitoring, remote modification of dose-fraction reminder timing, remote modification of fractionated-dose reminder timing, remote modification of dose-fraction mass delivery, and remote modification of fractionated-dose mass delivery.

Practice of the invention does not depend critically on the nature of the therapeutic biological or chemical agent being delivered. Suitable agents include, but are not limited to: Pilocarpine, artificial tears, latanoprostene, carboxymethylcellulose, propylene glycol, brinzolamide, nepafenac, travoprost, moxifloxacin, netarsudil, ketorolac, proparacaine, tetracaine, tobramycin, brimonidine, bimatoprost, lifitegrast, cyclosporine, gatifloxacin, besifloxacin, dexamethasone, loteprednol, prednisolone acetate, dorzolamide, timolol, azithromycin, bromfenac, levofloxacin, latanoprost, ketotifen, bepotastine, olopatadine, tetrahydrozoline, pheniramine maleate, sodium chloride (Muro), oxymetazoline, naphazoline, hydroxypropylmethylcellulose, cyclopentolate, phenylephrine, and tropicamide, as well as combinations or mixtures thereof.

It should be recognized that in some instances drugs with their primary pharmacologic effect serving as muscarinic agonists for certain receptor subclasses may act as antagonists in certain other cases including when more than one muscarinic agonist is used and there is competition for binding sites for the targeted receptor subclass, or when used alone in interactions with other muscarinic receptor subclasses.

It should also be recognized that the nature of the observed and predicted effects observed for fractional dosing with muscarinic or other designated receptor classes for agonists may also be seen for other therapeutic agents whose primary mode of agent is as antagonists not agonists for the designated receptor including atropine, homatropine, tropicamide, cyclopentolate, scopolamine and related compounds for the treatment of ocular disorders including myopia and related conditions.

1. A programmable handheld dispenser for delivering a microdose of a medication to a patient, the handheld dispenser comprising:
a container configured to hold the medication and having an aperture,
wherein the programmable handheld dispenser is configured to eject a fraction of a determined total dose volume of the microdose in two or more determined microdose-fractions, each microdose-fraction having a volume between about 1 μl and 20 μl,
wherein the handheld dispenser is programmed to deliver the two or more determined microdose-fractions;
wherein a therapeutic effect is increased by delivering the medication in the microdose fractions over delivering the total dose volume in a single dose; and
wherein the programmable handheld dispenser is configured to emit microdose fractions of the medication from the container through the aperture when one or more pulses is exerted onto the liquid in the container.

2. The programmable handheld dispenser of claim 1, wherein the programmable handheld dispenser is configured to provide a dose-fraction signal when a next microdose-fraction is due to be delivered.

3. The programmable handheld dispenser of claim 1, wherein the programmable handheld dispenser is configured to provide a total-dose signal when a next total-dose is due to be delivered.

4. The programmable handheld dispenser of claim 1, wherein the medication is a liquid ophthalmic agent for topical application to an eye of the patient.

5. The programmable handheld dispenser of claim 1, wherein the dispenser is configured for wireless communication with a remote device, wherein the remote device provides one or more of remote dose monitoring, remote modification of microdose-fraction reminder timing, remote modification of fractionated-dose reminder timing, remote modification of dose-fraction mass delivery, and remote modification of fractionated-dose mass delivery.

6. The programmable handheld dispenser of claim 1, wherein the microdose fractions are ejected as a stream of micro-droplets.

7. The dispenser of claim 1, wherein the programmable handheld dispenser is programmed to eject one or more subsequent microdose fractions at a determined time interval.

8. The programmable handheld dispenser of claim 7, wherein the time interval is less than a time interval at which an ocular surface returns to equilibrium.

9. A programmable handheld dispenser for delivering a microdose of a medication to a patient, the handheld dispenser comprising:
a container configured to hold the medication and having an aperture;
a valve configured to seal the aperture;
a motor;
an electronic circuit configured to generate and transmit an electric pulse or wave form to the motor, wherein the determined microdose-fractions are emitted from the container through the aperture when one or more pulses is exerted onto the liquid in the container, thereby opening the valve;
wherein the programmable handheld dispenser is programmed to eject a fraction of a determined total dose volume of the microdose in two or more determined microdose-fractions from the aperture, each microdose-fraction having a volume between about 1 μl and 20 μl; and
wherein a therapeutic effect is increased by delivering the medication in the microdose fractions over delivering the total dose volume in a single dose.

10. The programmable handheld dispenser of claim 9, further comprising a timer circuit that determines the length of time required to deliver the determined total dose volume.

11. The programmable handheld dispenser of claim 9, wherein the programmable handheld dispenser is in wireless communication with one or more remote devices, wherein the one or more remote devices provides one or more of remote dose monitoring, remote modification of microdose-fraction reminder timing, remote modification of fractionated-dose reminder timing, remote modification of dose-fraction mass delivery, and remote modification of fractionated-dose mass delivery.

12. The programmable handheld dispenser of claim 9, further comprising a membrane, wherein the motor is configured to vibrate the membrane such that the one or more pulses are delivered to the fluid via the membrane.

13. The programmable handheld dispenser of claim 9, wherein the motor is configured to vibrate or oscillate the container to deliver the one or more pulses to the fluid.

14. The programmable handheld dispenser of claim 9, wherein the medication is dispensed from the aperture as a stream of micro-droplets when the one or more pulses is exerted onto the fluid in the container.

15. The programmable handheld dispenser of claim 9, wherein the container is selected from a blow-fill-seal package and an ampoule.

16. The programmable handheld dispenser of claim 9, wherein the programmable handheld dispenser is configured to provide a dose-fraction signal when a next microdose-fraction is due to be delivered.

17. The programmable handheld dispenser of claim 9, wherein the programmable handheld dispenser is configured to provide a total-dose signal when a next total dose is due to be delivered.

18. The programmable handheld dispenser of claim 9, wherein the medication is a liquid ophthalmic agent for topical application to an eye of the patient.
